(19) United States
(12) Patent Application Publication
(10) Pub. No.: US 2015/0151281 A1
(43) Pub. Date: Jun. 4, 2015

(54) MULTI-METALLIC NANOMATERIALS FROM NI, AG, PD WITH PT'S CATALYTIC ACTIVITY

(71) Applicant: King Abdullah University of Science and Technology, Thuwal (SA)

(72) Inventors: Kuo-Wei HUANG, Thuwal (SA); Zhiping LAI, Thuwal (SA); Lei HU, Thuwal (SA)

(21) Appl. No.: 14/556,347

(22) Filed: Dec. 1, 2014

Related U.S. Application Data
(60) Provisional application No. 61/910,516, filed on Dec. 2, 2013.

(51) Int. Cl. B01J 23/89; C01B 3/04; B01J 37/16; H01M 8/04; B01J 35/02; C07C 5/03; B01J 37/34

(52) U.S. Cl. CPC B01J 23/892; C07C 5/03; C01B 3/04; B01J 37/343; H01M 8/04201; B01J 35/023; B01J 37/16; C07C 2521/18; C07C 2523/89

(57) ABSTRACT

A trimetallic catalyst that is a combination of nickel, silver and palladium metal is described. The trimetallic catalyst can be used to produce hydrogen and is useful as a replacement for platinum in hydrogenation reactions.

### CROSS REFERENCE TO RELATED APPLICATIONS

This application claims priority to U.S. Provisional Application Ser. No. 61/910,516, filed Dec. 2, 2013, the contents of which are incorporated herein in their entirety for all purposes

### FIELD OF THE INVENTION

The invention relates generally to a catalyst that is a combination of nickel, silver and palladium metal. The catalyst is useful as a replacement for platinum in hydrogenation reactions.

### BACKGROUND OF THE INVENTION

Hydrogen is considered an ideal secondary fuel and energy carrier with a high energy density by weight and it can be used in proton exchange membrane (PEM) fuel cells to produce electrical power for vehicles and electronic devices.^{[1]} Since hydrogen is a low density gas and flammable, how to store and use it safely becomes a major challenge.^{[2]} Current materials such as complex hydrides^{[3]} and chemical hydrides^{[4]} have been used for hydrogen storage, but most of these materials have low volumetric and/or gravimetric capacity which cannot meet the ideal requirement proposed for the on-board hydrogen storage system by the US department of energy (volumetric capacity>82 g/L and gravimetric capacity>9 wt. %).^{[5]} Boron hydrides are promising materials for this purpose,^{[6]} and in particular, ammonia borane (NH₃BH₃), denoted as AB) is one of the most studied boron hydrides as it stores 19.5 wt. % of hydrogen.^{[7]} Hydrogen release from AB is typically achieved through two pathways: thermolysis^{[8]} and hydrolysis.^{[9]} Thermal dehydrogenation of AB has been widely investigated, but it needs relatively higher temperatures to generate hydrogen completely. Hydrolysis of AB is a different approach and hydrogen can be released at ambient temperatures in the presence of suitable catalysts. Noble metal materials as the catalysts for hydrolysis of AB were first reported by Xu.^{[9a]} Pt and Rh supported on γ-Al₂O₃ has shown high catalytic activity (TOF=208 and 128 mol H₂ mol_{cat}⁻¹ min⁻¹, respectively).^{[10]} As the price of novel metals reached historical highs, the search for alternative materials with similar activities yet much lower cost is an important aspect for industrial and sustainable possibilities. Some transition metals like Co, Ni and Cu based catalysts were also tested, and in some cases, nearly stoichiometric amount of hydrogen was produced in the AB hydrolysis system with good catalytic activity.^{[11]} These catalysts are economical and recyclable, although the TOF value is lower than those of Pt and Rh based nanomaterials.

Bimetallic nanomaterials usually show enhanced catalytic activity, selectivity and stability versus their monometallic counterparts,^{[12]} and there have been a number of bimetallic nanomaterials used as the catalysts for hydrogen generation from AB solutions. The superior catalytic activity is rationalized by the synergistic and bi-functional effects^{[13]}. Ni and Co are the most utilized first-row transition metals which have been alloyed with noble metals (Au, Pt, Pd). These alloy nanoparticles can enhance the catalytic performance and reduce the consumption of the noble metals resulting in a viable approach for the development of low-cost catalysts. Liu and co-workers compared the catalytic activity of NiPt and NiPd nanomaterials and found that NiPt is more efficient than NiPd, and its monometallic counterparts (Ni or Pt).^{[13c]} The Xu group used metal-organic frameworks (MOFs) to immobilize AuNi nanoparticles and used them as the catalyst for AB hydrolysis with excellent catalytic performance (TOF=66.2 mol H₂ mol_{cat}⁻¹ min⁻¹) than those of monometallic Au and Ni immobilized by MOFs.^{[13d]} Trimetallic nanomaterials also show enhanced catalytic activity in hydrolysis of AB and some core-shell nanoparticles were synthesized and examined. [14]

Therefore, a need still exists for catalysts that overcome one or more of the current disadvantages noted above.

### BRIEF SUMMARY OF THE INVENTION

The present invention surprisingly provides an efficient trimetallic catalyst that is a combination of nickel, silver and palladium metal. The catalyst is useful as a replacement for platinum and or palladium in the generation of hydrogen from, for example, ammonia borane. The catalysts can also be used in hydrogenation reactions where platinum or palladium are typically utilized.

The synthesis of a trimetallic catalyst amalgam supported on carbon (NiAgPd/C) by reduction of the corresponding metal salts under mild reaction conditions is described herein. This trimetallic catalyst, as a nanomaterial in one embodiment, shows excellent catalytic activity and stability toward hydrogen generation though AB hydrolysis at room temperature (21° C.), comparable to those of Pt-based catalysts.

In one aspect, the trimetallic amalgam is formed with nickel, silver and palladium metal in a ratio of about 1 to about 1 to about 1. These ratios can be determined by ICP-MS.

In another aspect, the trimetallic catalyst described herein comprises nanoparticles of from about 1 to about 10 nm.

In another aspect, the trimetallic catalyst described herein has an X-ray diffraction pattern that provides a face-centered cubic (fcc) structure.

In still another aspect, the trimetallic catalyst has no detectable impurities as determined by XRD, XPS and/or EDX.

In another aspect, the trimetallic catalysts described herein are provided on a solid support, such as carbon.

NiAgPd trimetallic catalysts (amalgams or alloys) are successfully synthesized by in-situ reduction of Ni, Ag and Pd salts. This can be performed on the surface of carbon. In one aspect, nanoparticles are obtained. The catalytic activity of the trimetallic catalysts were examined in ammonia borane (NH₃BH₃) hydrolysis to generate hydrogen gas. The nanomaterial supported on carbon exhibited a higher catalytic activity than its monometallic and bimetallic counterparts and a stoichiometric amount of hydrogen was produced with a high generation rate. These hydrogen production rates were investigated in different concentrations of NH₃BH₃ solutions, including in borates saturated solution, showing little influence of the concentrations on the reaction rates. The hydrogen production rate can reach 3.6-3.8 mol H₂ mol_{cat}⁻¹ min⁻¹ at room temperature (21° C.). The activation energy and TOF value are 38.36 kJ mol⁻¹ and 93.8 mol H₂ mol_{cat}⁻¹ min⁻¹, respectively, comparable to those of Pt based catalysts. The present catalyst also exhibits excellent chemical stability, and no significant morphology change was observed from TEM after reaction. Using the trimetallic catalyst for continuously hydrogen generation, the hydrogen production rate can be kept after generating 6.2 L hydrogen with over 10,000 turnovers with a TOF value of 90.3 mol H₂ mol_{cat}⁻¹ min⁻¹. This high catalytic performance makes it an exciting candidate to generate hydrogen for portable PEM fuel cells.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 is a plot of time vs the ratio of hydrogen and AB in the presence of Ni, Ag, Pd based catalysts at 21° C. (nₘₑₜₐₗ: n_{AB}=0.012).

FIG. 2A demonstrates that TOF values were relatively stable (approx. 90 mol H₂ mol_{cat}⁻¹ min⁻¹), and no significant change was observed from 0.1 to 8.4 mol/L AB aqueous solutions.

FIG. 2B provides that a high TOF value was obtained (93.8 mol H₂ mol_{cat}⁻¹ min⁻¹) from the NiAgPd/C catalyst.

FIG. 2C provides that higher TOF values were obtained by increasing the reaction temperatures.

FIG. 3A demonstrates hydrogen generation from an AB aqueous solution in the presence of NiAgPd/C catalyst (nₘₑₜₐₗ: n_{AB}=0.012) at 20, 35, 50, 65 and 80° C.

FIG. 3B is the Arrhenius plot of Ink vs (1/T) from FIG. 3A.

FIG. 4A is a plot of time vs the ratio of hydrogen and AB in the presence of NiAgPd/C catalyst during a five cycle reusability test (nₘₑₜₐₗ: n_{AB}=0.012) at 21° C.

FIG. 4B provides the TOF values from the reusability test.

FIGS. 5A and 5B provide TEM images before (A) and after (B) five catalytic cycles (insert: the distribution of the nanoparticles).

FIG. 6A is an XRD pattern of a survey of NiAgPd/C.

FIG. 6B is an XPS spectrum of Ag 3d of NiAgPd/C.

FIG. 6C is an XPS spectrum of Pd 3d of NiAgPd/C.

FIG. 6D is an XPS spectrum of Ni 2p of NiAgPd/C.

FIG. 7 provides the XRD pattern of NiAgPd/C.

FIG. 8 provides the XRD pattern of NiAgPd/C compared with those of their counterparts.

FIGS. 9A, 9B and 9C provide STEM image (A) and EDX results (B, C) of NiAgPd/C at different positions.

FIG. 10 provides a GC-MS of cyclohexene before hydrogenation with NiAgPd/C.

FIG. 11 is a GC-MS of the reaction materials after hydrogenation of cyclohexene using NiAgPd/C as the catalyst. Only cyclohexane was noted.

FIG. 12 provides the comparison of gas generation using H₂O and D₂O as the solvents, where for D₂O, only the release of 2.8 equiv. of hydrogen was observed.

FIG. 13 provides XPS results of Ni after fitting.

### DETAILED DESCRIPTION

In the specification and in the claims, the terms “including” and “comprising” are open-ended terms and should be interpreted to mean “including, but not limited to . . . ” These terms encompass the more restrictive terms “consisting essentially of” and “consisting of”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Platinum (Pt) is a precious metal which is used as a catalyst in important chemical reactions, such as hydrogen generations from ammonia borane (AB), hydrogenations, electrocatalysis and so on. As the price of Pt reached its historical height, the search for alternative materials with similar activities, yet much lower cost, is important for commercialization and for a sustainable future. Numerous research effects worldwide have been devoted to the development of new materials which can be used instead of Pt. Since multi-metallic nanomaterials often show enhanced catalytic activity and selectivity than its monometallic counterparts, and the synthesis of such materials decreases the contents of novel metals, they become viable approaches for non-Pt catalyst development. Described herein are tri-metallic catalysts which are alloys and in certain aspects, nanomaterials, from Ni, Ag, Pd. These novel trimetallic catalysts show they are excellent catalysts for hydrogen generation through AB hydrolysis comparable to those of Pt based catalysts. The production cost of these new trimetallic catalysts is less than 7% of those of Pt catalysts.

In one aspect, the trimetallic NiAgPd catalyst was synthesized by using co-reduction of metal salts in water without any surfactants. Generally, active carbon was first ultrasonically dispersed in water and stirred under an argon atmosphere. The solution(s) of the solution of three metal salts were/was then added to dispersed active carbon under argon before treatment of with a reducing agent, such as sodium borohydride, for example, a fresh NaBH₄ aqueous solution to reduce the metal salts. The supported nanomaterial was then conveniently obtained through filtration.

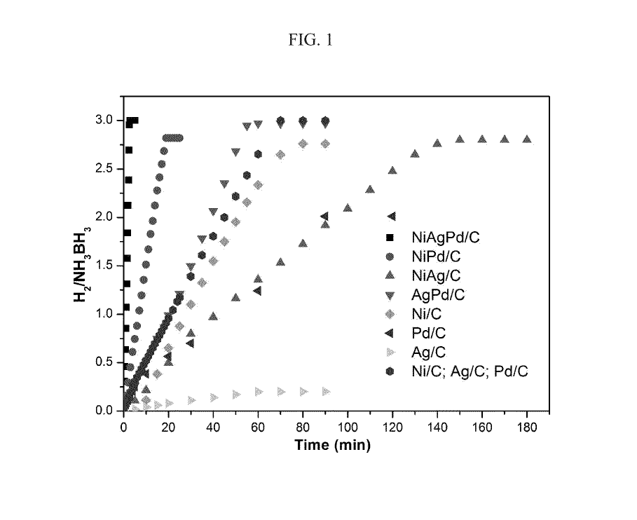

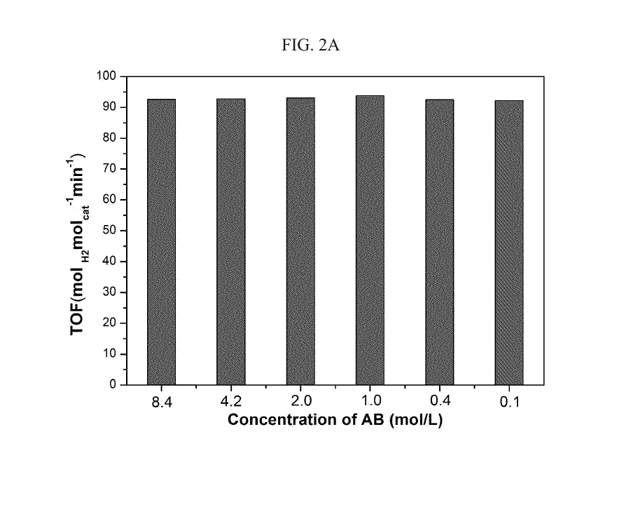

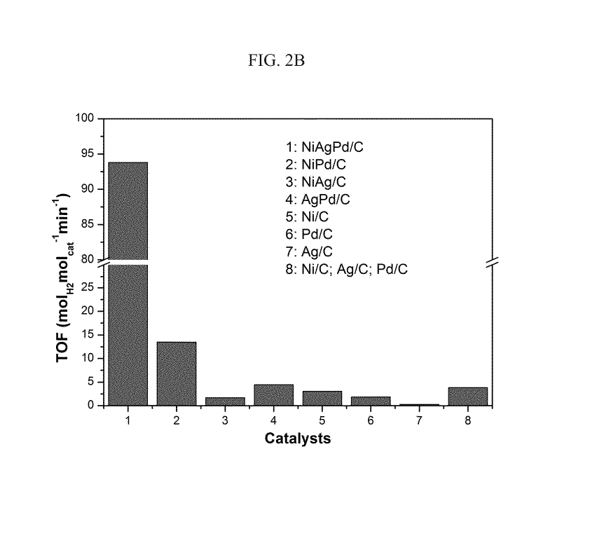

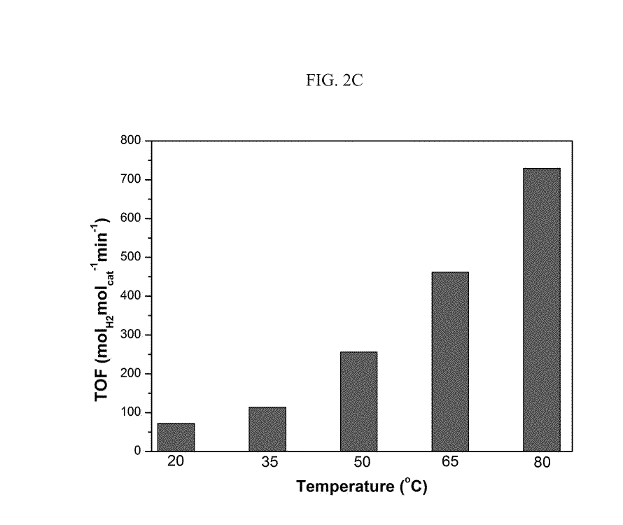

The catalytic activities of the resulting NiAgPd/C and monometallic (Ni/C, Ag/C, and Pd/C), bimetallic (NiPd/C, NiAg/C, and AgPd/C) counterparts were investigated in hydrogen production reaction from AB aqueous solution at room temperature (FIG. 1). NiAgPd/C exhibits much improved catalytic activity in comparison to the mono-metallic and bi-metallic counterparts which were synthesized through similar processes. 3.0 equivalents of hydrogen were produced from AB aqueous solution when NiAgPd/C or AgPd/C was used as the catalyst, indicating complete hydrogen generation. A high TOF value was obtained (93.8 mol H₂ mol_{cat}⁻¹ min⁻¹) from the NiAgPd/C catalyst (FIG. 2B). This value is also comparable to those reported for Pt and Rh catalysts:^{[10]} It is evidenced that Ni can enhance the catalytic activity by comparing the catalytic activity of NiAgPd/C with that of AgPd/C and the catalytic activity of NiPd/C with that of Pd/C. From its monometallic counterparts, Ni/C shows higher catalytic activity than Pd/C and Ag/C and 2.8 equivalents of hydrogen were generated in 80 min. The catalytic activity of NiPd/C is higher than those of AgPd/C and AgNi/C and 2.8 equivalents of hydrogen were generated in 18 min. The catalytic activity of NiAgPd/C was further compared with the mechanical mixture of its monometallic counterparts (molar ratio of Ni, Ag and Pd is the same as that in NiAgPd/C). The mixture shows a higher catalytic activity than those of individual catalysts and 3 equivalents of hydrogen were generated in 70 min. However, the catalytic activity is lower (3.85 mol H₂ mol_{cat}⁻¹ min⁻¹) when compared with the NiAgPd/C catalyst.

Hydrogen production rates at different AB concentrations were also studied in the presence of NiAgPd/C catalyst. As shown in FIG. 2A, TOF values were relatively stable (approx. 90 mol H₂ mol_{cat}⁻¹ min⁻¹), and no significant change was observed from 0.1 to 8.4 mol/L AB aqueous solutions. These results indicated that the catalytic activity was not influenced by the concentration of the AB solution. From the gas production curve in the presence of NiAgPd/C, the ratio of the volume of the hydrogen versus time is constant, consistent with the observation that the catalytic activity is not concentration dependent. At high concentration of 8.4 mol/L, some solids were formed after the reaction. These solids were later confirmed to be borates due to their limited solubility in water.^{[9b]} The catalytic activity of NiAgPd/C was further examined using a borates saturated solution under the similar reaction condition. 3.0 equivalents of hydrogen gas were generated and the TOF value was 91.1 mol H₂ mol_{cat}⁻¹ min⁻¹, close to those obtained from lower AB concentrations. These results suggest that NiAgPd/C is suitable for continuously generating hydrogen from AB aqueous solutions by removing the borates sediment in the reaction and the catalytic activity will not be significantly affected by changing the concentration of the AB solution.

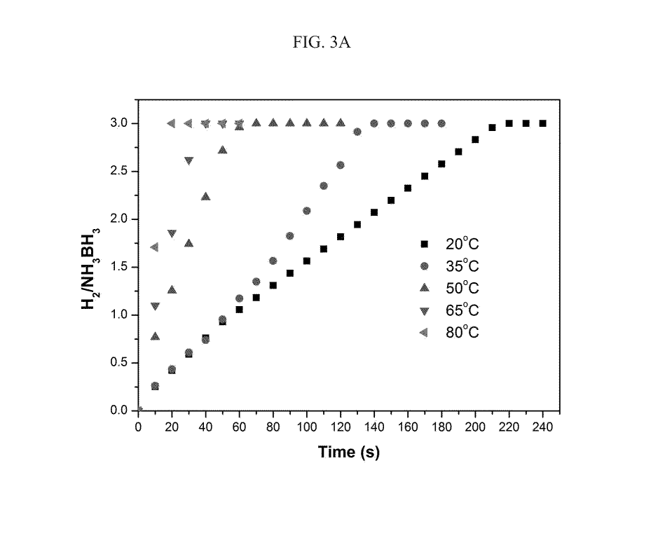

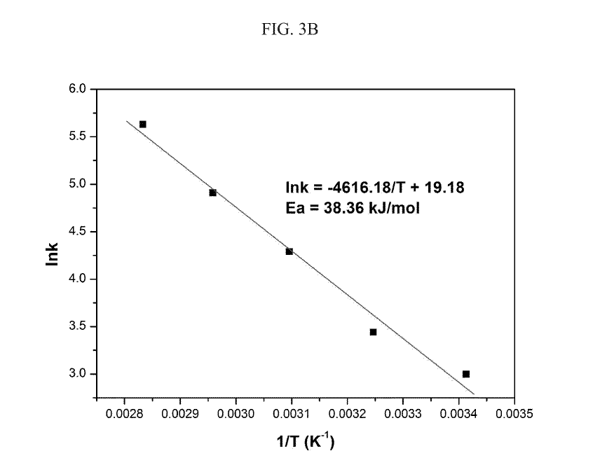

As catalytic rate can be generally accelerated by increasing the reaction temperature, different temperatures for catalytic hydrogen generation were also examined. Hydrogen can be generated completely in a shorter time in the presence of the NiAgPd/C catalyst when increasing the reaction temperature. At 80° C., 3.0 equivalents of hydrogen was obtained in 17 seconds and the corresponding TOF value could reach 729.5 mol H₂ mol_{cat}⁻¹ min⁻¹ (FIG. 2C). Under these conditions (NiAgPd/C, 80° C.), hydrogen could be generated continuously by re-adding AB and water into the solution, and the hydrogen production rate was about 29.8 mol H₂ h⁻¹ g_{cat}⁻¹. These parameters indicate that in order to produce 1 kWh of electricity in a proton exchange membrane (PEM) fuel cell system, only about 0.9 g of NiAgPd/C catalyst would be sufficient. Even at room temperature (21° C.), 7.3 g of NiAgPd/C catalyst would be sufficient to provide hydrogen for the PEM fuel cell system (the amount of NH₃BH₃ needed is 0.27 kg^{[10]}, assume the operation circuit voltage is 0.7V for this standard PEM fuel cell). In FIG. 3, hydrogen production rates are nearly constants at different temperatures. These rate constants (κ) can be used to calculate the activation energy for NiAgPd/C catalytic hydrogen generation reaction.^{[13a,c,15]} With these rate constants obtained at different temperatures, Arrhenius plot (ln κ vs 1/T) for the catalytic hydrogen production reaction gave the activation energy of 38.4 kJ/mol. By comparing the catalytic activity with Ni, Ag, and Pd based catalysts (Table 1), NiAgPd/C shows the optimal catalytic activity comparable to those of Pt based catalysts (Pt/C, Pt black and PtO₂ catalysts).^{[9b]}

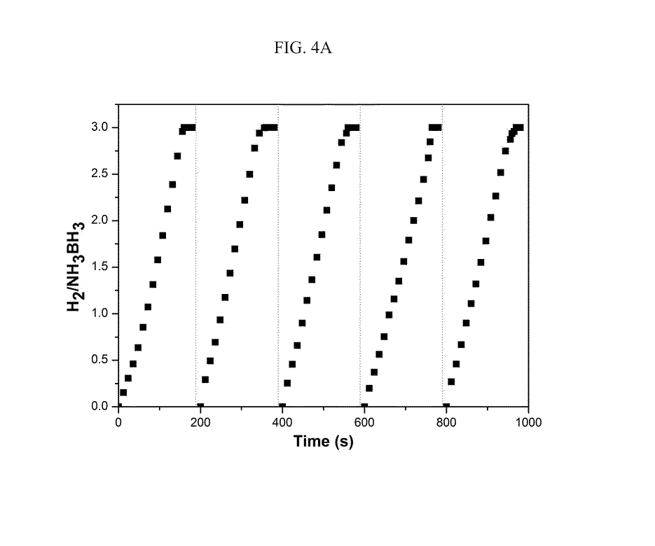

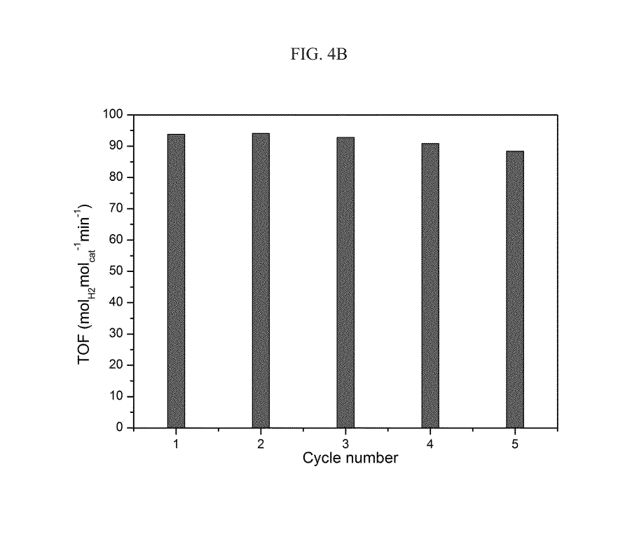

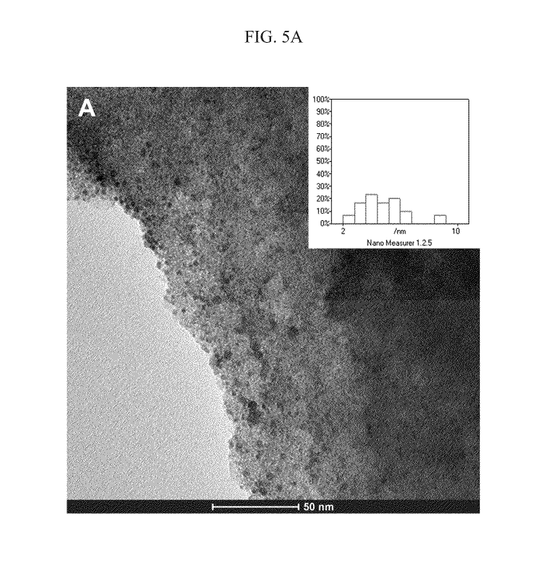

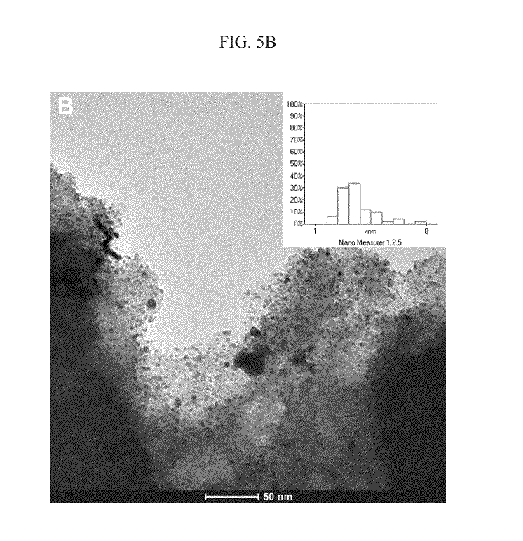

The stability and reusability of the as-synthesized NiAgPd/C catalyst was also tested in AB hydrolysis at room temperature. The volume of hydrogen was measured at different times, and re-addition of AB solutions (2.0 mmol) was carried out after the complete hydrogen generation from the prior addition. The first four cycles were obtained continually and they could all be finished in 160 s (FIG. 4A). In order to evaluate the stability of the catalyst, the reaction mixture was further stirred at room temperature for 24 hours after the fourth cycle was completed. Another one equivalent of AB (2.0 mmol) was then added, and complete hydrogen generation was achieved in 170 s with the TOF value remaining approximately the same (88.4 mol H₂ mol_{cat}⁻¹ min⁻¹, FIG. 4B). The reaction solution was analyzed after the hydrogen generation, and only 0.14% of the Ni leakage was detected by ICP-MS. The size and morphology were not significantly changed during the reaction (FIG. 5B). The lifetime of NiAgPd/C was evaluated by adding AB into the reaction mixture continually and the turnover number (TON) of 500,000 was achieved without obvious loss of the catalyst's activity. These results indicate that the NiAgPd/C catalyst can be a promising non-Pt catalyst for hydrogen generation from AB hydrolysis.

TABLE 1
H₂ generation from aqueous AB solution catalyzed by
Ni, Ag, Pd, Pt based catalysts at room temperature.

| | | TOF | | |
|---|---|---|---|---|
| | metal/AB | (mol H₂ mol_{cat}⁻¹ | Eₐ | |
| Catalyst | (mol/mol) | min⁻¹) | (kJ/mol) | ref |
| 2 wt % Pt/C | 0.018 | 111 | — | 9b |
| 20 wt % Pt/C | 0.018 | 83.34 | — | 9b |
| Pt black | 0.018 | 13.89 | — | 9b |
| PtO₂ | 0.018 | 20.8 | — | 9b |
| Ni in starch | 0.1 | 5 | — | 16 |
| Bare Ni NPs | 0.1 | 2.5 | — | 11a |
| PVP-Ni NPs | 0.1 | 3 | — | 11a |
| Ag/Ni/G | 0.05 | 77.0 | 49.56 | 15b |
| Ag/C/Ni | 0.022 | 5.32 | 38.91 | 17 |
| Ni/ZIF-8 | 0.016 | 14.2 | — | 11g |
| Pd/HAP | 0.02 | 6.8 | 55 ± 2 | 18 |
| Pd black | 0.018 | 0.67 | — | 9a |
| CoPd/C | 0.024 | 22.7 | 27.5 | 13b |
| NiAgPd/C | 0.012 | 93.8 | 38.36 | Disclosed |
| | | | | herein |

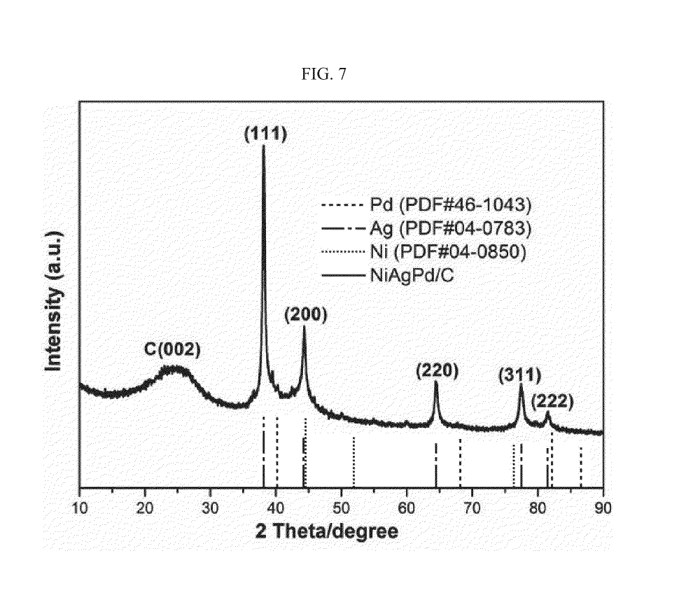

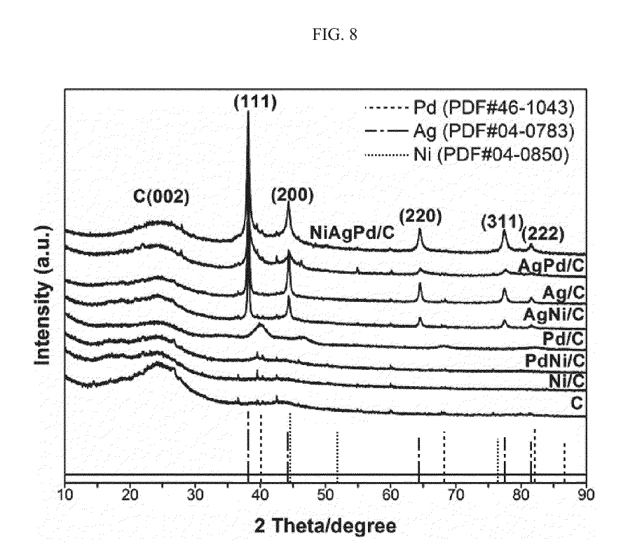

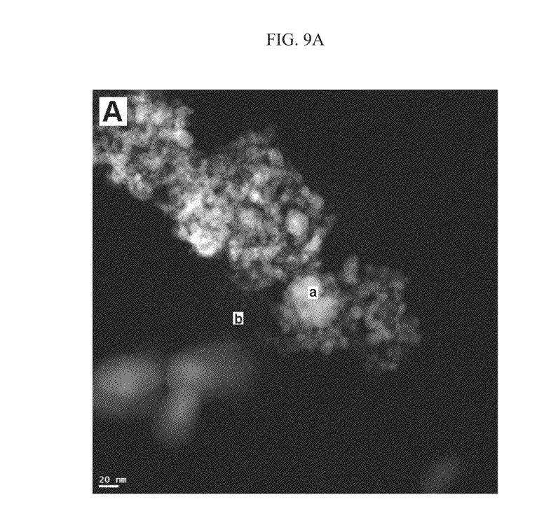

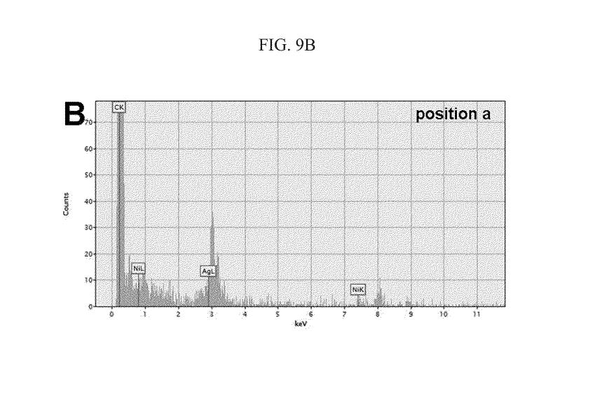

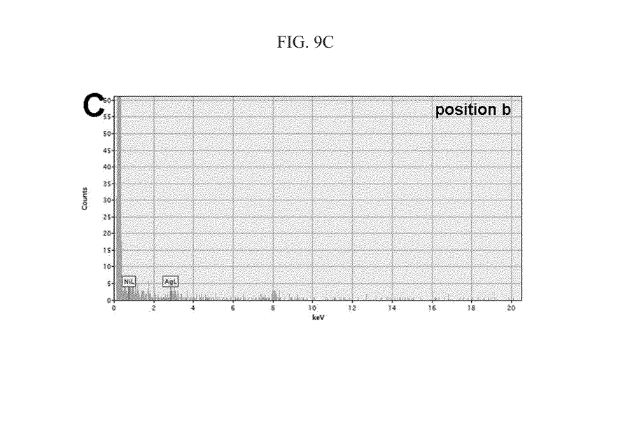

The NiAgPd/C trimetallic catalyst was characterized by ICP-MS to determine the ratio of these three metals and their loading. The results showed that the ratio of Ni:Ag:Pd was approximately 1:1:1 with the metal loading of approximately 6.2 wt. %, consistent with the expected outcomes from experimental conditions. The nanoparticles were well-dispersed on the surface of carbon and the sizes were in a range of 3-8 nm from TEM images and the nanoparticles size distribution (FIG. 5A). The NiAgPd/C catalyst showed good stability and no significant changes were observed in nanoparticle size and morphology after 5 cycles (FIG. 5B). Scanning TEM (STEM) and energy dispersive X-ray (EDX) were also used to characterize the structure and components of the catalyst (FIGS. 9A, 9B and 9C). As the nanoparticles were small and supported on carbon, the analysis was interfered by carbon and only weak peaks of Ni, Ag, and Pd were observed. The X-ray diffraction (XRD) pattern exhibited an fcc structure close to that of metallic Ag (FIG. 7) and no impurity was observed. Together with the ICP-MS results, it was concluded that NiAgPd alloy nanoparticles were formed to give a pure fcc structure:^{[19]}

Not to be limited by theory, since the atomic radius of Ag (0.144 nm) is bigger than those of Pd (0.138 nm) and Ni (0.125 nm),^{[20]} it is conceivable that the fcc alloy structure is close to the fcc structure of Ag, and Ni and Pd exist in this Ag-like fcc structure. Strain effects on Ni, Pd are thus expected in these nanoalloy nanoparticles resulting in a higher reactivity.^{[21]}

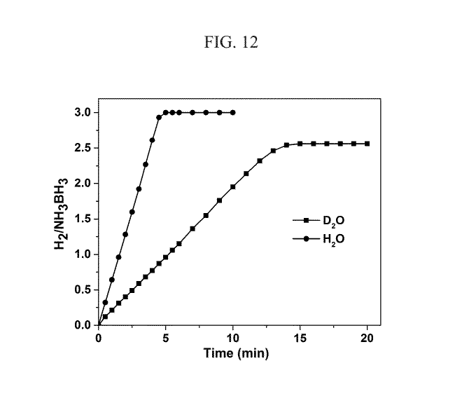

This expectation was confirmed by experimental results (FIG. 1) that NiAgPd/C has a higher catalytic activity than NiPd/C, Ni/C and Pd/C. Density functional theory (DFT) calculations were used to support this strain effect in the NiAgPd system. In comparative reactions, the gas generation rate was firstly compared using D₂O and H₂O as the solvent, the gas generation rate in H₂O is about three times than in D₂O which demonstrates that the water decomposition is the rate determining step (FIG. 12). Therefore, hydrogen atom adsorption can be used to indicate the catalytic activity^{[23]}. The adsorption energy was firstly compared between Pd and Pd with Ag lattice. Stronger hydrogen adsorption was obtained on Pd with Ag lattice (2.97 eV) than Pd (2.90 eV) which demonstrated that hydrogen will be generated easier in Pd with Ag lattice system. Meanwhile, a higher hydrogen adsorption was observed in NiAgPd system (3.00 eV) than NiPd (2.75 eV), Ni (2.80 eV) and Pd (2.90 eV) system which is in good agreement with our experiment results that NiAgPd shows the highest catalytic activity in hydrogen generation from AB hydrolysis.

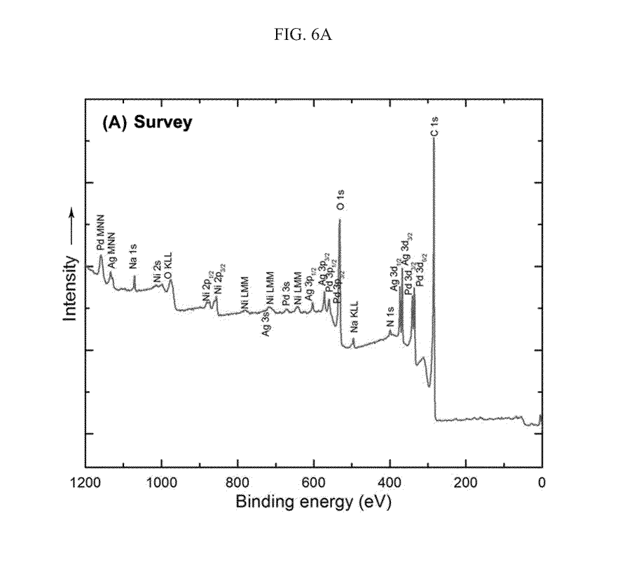

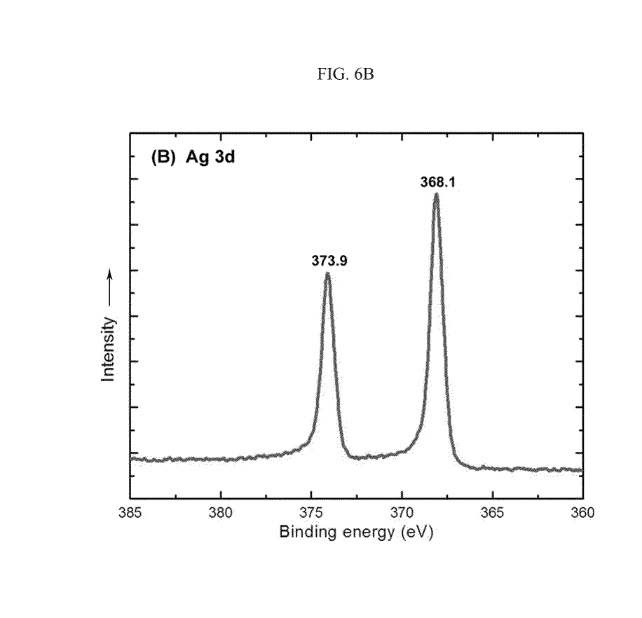

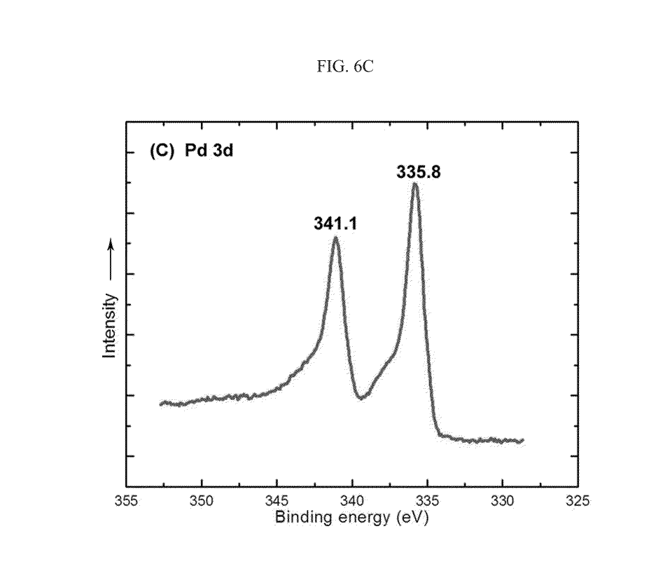

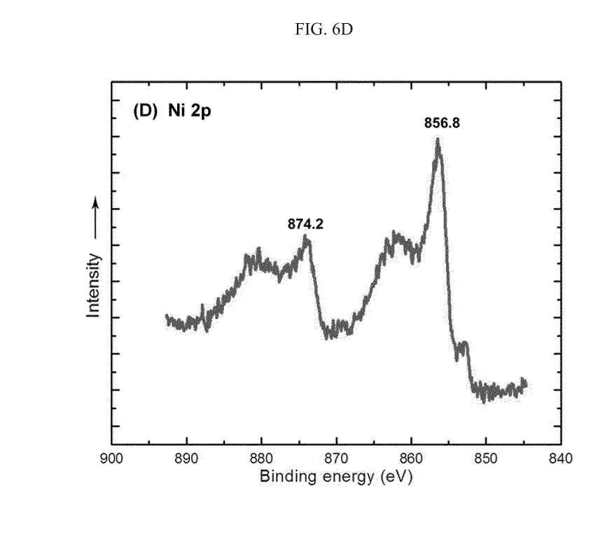

FIG. 6B shows two peaks of Ag 3d of the catalyst in the XPS (368.1 and 373.9 eV), in good agreement with the values for Ag⁰. Peaks with binding energies of 335.8 and 856.8 eV in FIGS. 6C and 6D are likely attributed to Pd 3d_{5/2} of the Pd⁰ and Ni 2p_{3/2} of Ni⁰ respectively. Compared with the NiPd nanoalloy, the Pd 3d peak shifted to a lower value and the Ni 2p peak shifted to a higher value in the NiAgPd system.^{[22]} These shifts from XPS peaks confirmed that electrons have been transferred from Ni to Pd which likely enhance the catalytic activity.^{[19],[23]}

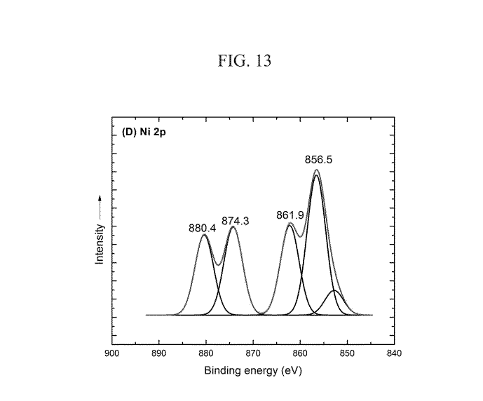

Peaks in FIG. 6D with binding energies of 856.8 and 874.2 eV represent oxidized Ni and a small peak at 852.9 eV corresponds to Ni⁰ (15%) (FIG. 13). These results indicate that Ag, Pd are stable, whereas the Ni may be transferred to outside and oxidized to certain degree after the catalyst was synthesized and during its application. Therefore, the slight decrease in the catalytic activity after stirring under air for 24 h may be attributed to the oxidation of the Ni in that nanomaterial. According to the nature of electronegativity, the electrons may transfer from Ni (1.91) and Ag (1.93) to Pd (2.20) which can make the Pd surface negative with high catalytic activity.^{[24]} This similar phenomenon was observed in CoAuPd/C catalyst for the formic acid decomposition.

NiAgPd nanoparticles supported on carbon have been designed and successfully prepared. These nanomaterial catalysts exhibit high catalytic activity and stability towards hydrogen generation from NH₃BH₃ hydrolysis at room temperature. Stoichiometric amounts of hydrogen were produced under a constant TOF of 93.8 mol H₂ mol_{cat}⁻¹ min⁻¹. The activation energy was determined to be 38.36 kJ/mol. The process was proved to be efficient in a wide range of concentrations of the NH₃BH₃ solution to afford stoichiometric amounts of hydrogen with a low catalyst/NH₃BH₃ ratio (0.012). Hydrogen can be continually produced by adding fresh NH₃BH₃ solutions. This catalyst provide 10216 turnovers by continuously adding NH₃BH₃ solution into the reaction system and the catalytic activity was kept (TOF=90.3 mol H₂ mol_{cat}⁻¹ min⁻¹). The trimetallic nanomaterials provide a new approach to prepare the catalyst for highly efficient portable hydrogen generation system. When using this generated hydrogen to produce 1 kWh of electricity in a proton exchange membrane (PEM) fuel cell system, about 7.3 g of NiAgPd/C catalyst would be sufficient as the catalyst to generate hydrogen at room temperature (the amount of NH₃BH₃ needed is 0.27 kg, assuming the operation circuit voltage is 0.7V for a standard PEM fuel cell).

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The following paragraphs enumerated consecutively from 1 through 24 provide for various aspects of the present invention. In one embodiment, in a first paragraph (1), the present invention provides a trimetallic catalyst comprising a combination of nickel, silver and palladium metal.

2. The catalyst of paragraph 1, wherein the nickel, silver and palladium metal is in a ratio of about 1 to about 1 to about 1, more particularly from about 0.1:1:1, 0.2:1:1, 0.3:1:1, 0.4:1:1, 0.5:1:1, 0.6:1:1, 0.7:1:1, 0.8:1:1, 0.9:1:1, 0.95:1:1, 0.098:1:1, 0.99:1:1, 1:1:1, 1:0.1:1, 1:0.2:1, 1:0.3:1, 1:0.4:1, 1:0.5:1, 1:0.6:1, 1:0.7:1, 1:0.8:1, 1:0.9:1, 1:0.95:1, 1:0.98:1, 1:0.99:1, 1:1:0.1, 1:1:0.2, 1:1:0.3, 1:1:0.4, 1:1:0.5, 1:1:0.6, 1:1:0.7, 1:1:0.8, 1:1:0.9, 1:1:0.95, 1:1:0.98, 1:1:0.99 and all values there between, including odd and even integers, for example, 0.11, 0.12, 0.13, 0.14, etc., 0.21, 0.22, 0.23, 0.24, etc. through 0.99.

3. The catalyst of paragraph 2, wherein the ratio is determined by ICP-MS.

4. The catalyst of any of paragraphs 1 through 3, wherein the catalyst comprises nanoparticles of from about 1 to about 10 nm.

5. The catalyst of any of paragraphs 1 through 4, wherein an X-ray diffraction pattern comprises a face-centered cubic (fcc) structure.

6. The catalyst of paragraph 5, wherein no impurities are included.

7. The catalyst of any of paragraphs 1 through 6, further comprising the catalyst on a solid support.

8. The catalyst of paragraph 7, wherein the solid support is an inorganic oxide carrier.

9. The catalyst of paragraph 8, wherein the inorganic oxide carrier is a metal oxide comprising alumina, silica, titania or zirconia.

10. The catalyst of any of paragraphs 7 through 9, wherein the surface area of the support is at least 100 g/m² to at least 1500 g/m².

11. The catalyst of paragraph 7, wherein the solid support is activated carbon.

12. The catalyst of paragraph 11, wherein the activated carbon has a surface area of from about is at least 100 g/m² to at least 1500 g/m².

13. A method to produce hydrogen comprising the step of contacting ammonia borane represented by the chemical formula NH₃BH₃ with the catalyst of any of paragraphs 1 through 12.

14. A method to provide a hydrogen supply comprising supplying hydrogen generated by paragraph 13 to a fuel cell.

15. The method of paragraph 14, wherein the fuel cell is a polymer electrolyte fuel cell, phosphoric acid fuel cell, or a high temperature fuel cell.

16. The method of paragraph 13, wherein platinum can be replaced with the catalysts of any of paragraphs 1 through 12, wherein the catalyst is used for hydrogenation of materials.

17. A method to prepare a trimetallic catalyst comprising nickel, silver and palladium metal comprising the steps:

combining a nickel (II) salt, silver (II) salt and palladium (IV) salt, alone or in combination, wherein the salts can be halides, acetates, nitrates, etc. to provide a first mixture; and

treating the mixture with a reducing agent, such as sodium borohydride, lithium aluminum hydride, sodium cyanoborohydride, etc. to provide a second mixture to provide a trimetallic nickel, silver and palladium trimetallic catalyst.

18. The method of paragraph 17, further comprising treating the first mixture under ultrasonic conditions.

19. The method of either paragraph 17 or 18, further comprising treating the second mixture under ultrasonic conditions.

20. The method of any of paragraphs 17 through 19, further comprising the step of adding a solid support to either the first mixture or the second mixture.

21. A method to hydrogenate olefins comprising the step of treating an olefin with a NiAgPd catalyst in the presence of hydrogen.

22. The method of paragraph 21, wherein the catalyst is supported on an inorganic oxide or a carbon material.

23. The method of either of paragraphs 21 or 22, wherein the hydrogenation reaction is conducted under pressure.

24. The method of any of paragraphs 21 through 23, wherein the reaction is conducted in a solvent which is non-reactive to the catalyst and the hydrogen.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### EXAMPLES

### Experiment Details

Synthesis of NiAgPd/C Nanomaterials:

Active carbon (400.0 mg, surface area 450 m²/g) was dispersed in water (100.0 mL) by sonication for 30 min to get a well-dispersed active carbon water solution. To this solution under argon atmosphere was added a water solution of metal salts (Ni(OAc)₂.4H₂O, 44.8 mg; AgNO₃, 30.5 mg; Pd(NO₃)₂.xH₂O, 50.1 mg in 10.0 mL of H₂O). This reaction mixture was further ultrasonicated for 30 min before a fresh NaBH₄ aqueous solution (200.0 mg NaBH₄ in 10.0 mL of H₂O) was added dropwise with rigorous stirring. After the addition was completed, this mixture was stirred for 18 hr at room temperature. The resulting black precipitates were collected through filtration and dried in the vacuum oven at 120° C. for 24 hours (426.0 mg).

For comparison, AgPd/C, NiPd/C, NiAg/C, Ni/C, Pd/C and Ag/C were also prepared by the same method.

Hydrogen production by NiAgPd/C catalytic hydrolysis of AB aqueous solution: The catalytic activity of the catalysts was calculated by measuring the volume of the hydrogen produced by hydrolysis of AB in a water-filled gas buret system. The catalyst was first sealed in the reaction flask collect to the gas collection system. Then, AB aqueous solution was injected into this system under stirring. The volume of hydrogen gas generated from the system was recorded by measuring the displacement of water volume at different time (subtract the volume of the AB solution). When stoichiometric amounts of hydrogen were collected, equivalent of AB solution was re-added to test the reusability of the catalyst.

Characterization

ICP-MS: NiAgPd/C (11.9 mg) was added to the Nitric Acid (65% solution in water, 5.0 mL), and then heated to 80° C. for 30 min. This mixture was filtrated to remove carbon and the filtered carbon was washed with water for several times. The filtrates were combined and diluted to 100.0 mL (<5 ppm). This solution was then analyzed using ICP-MS (Perkin Elmer, Elan DRC-II) to get the metal loading (6.2 wt. %) and the molar ratio of the three metals (about 1:1:1).

XRD: The sample was analyzed directly using the XRD standard method (XRD, Bruker, D8 Advance, scan from 10 to 90 degrees, stepsize: 2 theta=0.0205283 degree). See FIG. 7.

XPS measurements were carried out using a monochromatic Al Kα X-ray source operating at 150 W. The survey and high-resolution spectra were collected at fixed analyzer pass energies of 160 and 20 eV, respectively. See FIGS. 6B, 6C and 6D.

STEM and EDX: The sample was dispersed in ethanol by sonication and added to the copper grid for the TEM and EDX analysis (Electron microscope 120 kV, Titan from FEI). FIGS. 9A through 9C.

The NiAgPd/C material is also an excellent catalyst for olefin hydrogenation (comparable to that of Pt/C).

Hydrogenation of cyclohexene using NiAgPd/C as the catalyst was examined.

NiAgPd/C (11.5 mg), cyclohexene (0.5 g) and methanol (5.0 mL) was added to the autoclave (25 mL). The autoclave was sealed and flashed with argon for 5 times, and then flashed and filled using hydrogen for 5 times. The hydrogen pressure was increased to 150 psi at room temperature (21° C.). The reaction solution was stirred at room temperature for 15 min. The solution was then analyzed by GC-MS, and only cyclohexane was observed as the single product.

NiAgPd/C (11.5 mg), cyclohexene (0.5 g) and methanol (5.0 mL) was added to the autoclave (25 mL). The autoclave was sealed and flashed with argon for 5 times, and then flashed and filled using hydrogen for 5 times. The hydrogen pressure was increased to 30 psi at room temperature (21° C.). The reaction solution was stirred at room temperature for 30 min. The solution was then analyzed by GC-MS, and only cyclohexane was observed as the single product.

GC-MS Analysis Condition:

Temperature procedure: (1) Initial temperature 80° C., hold 2 min. (2) Increase to 200° C. with a rate of 15° C./min. (3) Increase to 280° C. with a rate of 50° C./min, hold 2.5 min. (GC: Agilent technologies 7890A; MS: Agilent technologies, 5975C inert XL EI/CI MSD with Triple Axis Detector).

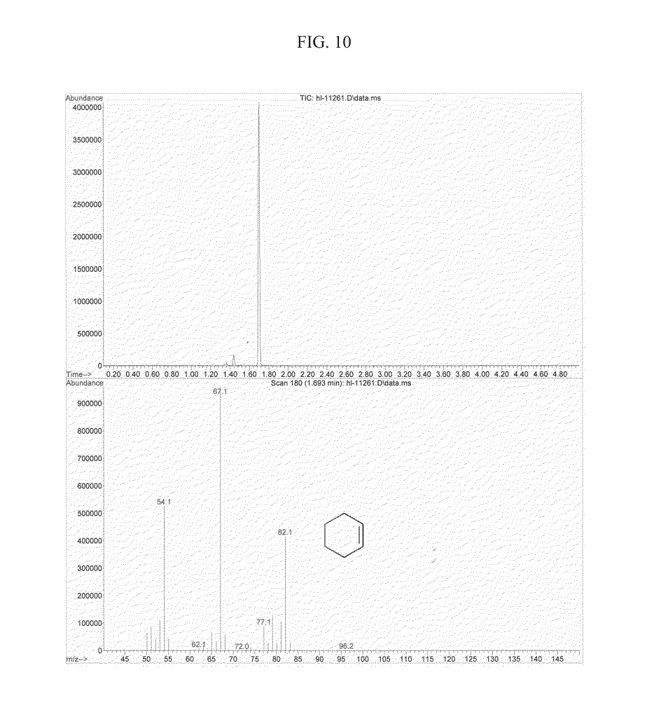

FIG. 10, before hydrogenation (cyclohexene).

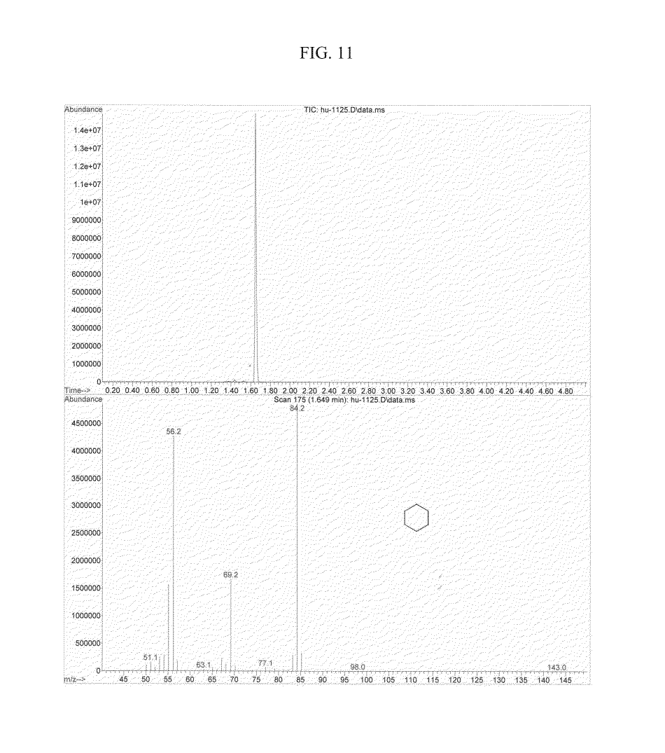

FIG. 11, after hydrogenation of cyclohexene using NiAgPd/C as the catalyst.

Calculation Section

Density functional theory (DFT)^{[1,2]} calculations were conducted employing CASTEP code^{[3,4]}. Ion-electron interactions and exchange-correlation functions are described by the ultrasoft pseudopotentials^{[5]} and the spin-polarized Perdew-Burke-Ernzerhof generalized gradient approximation (GGA-PBE)^{[6]}, respectively. A plane-wave cutoff energy of 400 eV was used and the convergence criterion for electronic selfconsistency is 5.0×10⁻⁷ eV/atom. The Broyden-Fletcher-Goldfarb-Shanno (BFGS) method^{[7]}, implemented in the CASTEP code, was employed to perform geometry optimizations.

A p(3×4) lateral super cell is built with a 4×3×1 k-point mesh constructed using the Monkhorst and Pack scheme [8]. The surfaces are modelled with a three-layer slab with a vacuum thickness of 14 Å. The bimetallic and trimetallic systems are designed with uniformly distribution of different types of metallic atoms on every layer. The position of all atoms except those in the bottommost layer are fully relaxed to satisfy the optimization criteria (1.0×10⁻⁵ eV/atom for energy change, 0.03 eV/Å for maximum force, 0.05 GPa for maximum stress and 1.0×10⁻³ Å for maximum displacement).

Adsorption Energies, E_{ad} (eV), of H on Mono-, Bi- and Trimetallic Surfaces.

| | Surfaces | | | | |
|---|---|---|---|---|---|
| | | | Pd with Ag | | |
| | Ni(111) | Pd(111) | lattice | NiPd | NiAgPd |
| E_{ad} (H) | 2.80/2.83^{a}, | 2.90/2.88^{b} | 2.97 | 2.75 | 3.00 |
| (eV) | 2.89^{b} | | | | |

^{a}Ref. 9,
^{b}Ref. 10

### REFERENCES

1 (a) P. P. Edwards, V. L. Kuznetsov, W. I. F. David, N. P. Brandon, Energy Policy 2008, 36, 4356; (b) H. J. Neef, Energy 2009, 34, 327; (c) G. W. Crabtree, M. S. Dresselhaus, M. V. Buchanan, Phys. Today 2004, 57, 39; (d) M. Ball, M. Wietschel, Int. J. Hydrogen Energy 2009, 34, 615.
2 L. Schlapbach, A. Züttel, Nature, 2001, 414, 353.
3 (a) S. Orimo, Y. Nakamori, J. R. Eliseo, A. Züttel, C. M. Jensen, Chem. Rev. 2007, 107, 4111; (b) F. Schüth, B. Bogdanović, M. Felderhoff, Chem. Commun. 2004, 2249.
4 (a)R. B. Biniwale, S. Rayalu, S. Devotta, M. Ichikawa, Int. J. Hydrogen Energy 2008, 33, 267; (b) E. Fakioglu, Y. Yürüm, T N. Veziroglub, Int. J. Hydrogen Energy 2004, 29, 1371.
5 (a) R. K. Ahluwalia, T. Q. Huang, J. K. Peng, Int. J. Hydrogen Energy 2012, 37, 2891; (b) M. Yadav, Q. Xu, Energy Environ. Sci. 2012, 5, 9698; (c) S. Satyapal, J. Petrovic, C. Read, G. Thomas, G. Ordaz, Catal. Today 2007, 120, 246.
6 (a) Z. Xiong, C. K. Yong, G. Wu, P. Chen, W. Shaw, A. Karkamkar, T. Autrey, M. O. Jones, S. R. Johnson, P. P. Edwards, W. I. F. David, Nat. Mater. 2007, 7, 138; (b) A. Züttel, A. Borgschulte, S. Orimo, Scripta Mater. 2007, 56, 823.
7 (a) B. Peng, J. Chen, Energy Environ. Sci. 2008, 1, 479; (b) R. J. Keaton, J. M. Blacquiere, R. Tom Baker, J. Am. Chem. Soc. 2007, 129, 1844; (c) F. H. Stephens, R. Tom Baker, M. H. Matus, D. J. Grant, D. A. Dixon, Angew. Chem. Int. Ed. 2007, 46, 746; (d) T. B. Marder, Angew. Chem. Int. Ed. 2007, 46, 8116.
8 (a) A. Gutowska, L. Li, Y. Shin, C. M. Wang, X. S. Li, J. C. Lineha, R. S. Smith, B. D. Kay, B. Schmid, W. Shaw, M. Gutows, T. Autrey, Angew. Chem. Int. Ed. 2005, 44, 3578; (b) U. B. Demirci, S. Bernard, R. Chiriac, F. Toche, P. Miele, J. Power Source 2011, 196, 279; (c) M. E. Bluhm, M. G. Bradley, R. Butterick III, U. Kusari, L. G. Sneddon, J. Am. Chem. Soc. 2006, 128, 7748.
9 (a) M. Chandra, Q. Xu, J. Power Source 2006, 156, 190; (b) Q. Xu, M. Chandra, J. Alloys Compd. 2007, 446-447, 729.
10 M. Chandra, Q. Xu, J. Power Source 2007, 168, 135.
11 (a) T. Umegaki, J. M. Yan, X. B. Zhang, H. Shioyama, N. Kuriyama, Q. Xu, Int. J. Hydrogen Energy 2009, 34, 3816; (b) M. Rakap, S. Özkar, Int. J. Hydrogen Energy 2010, 35, 3341; (c) B. H. Liu, Z. P. Li, S. Suda, J. Alloys Compd. 2006, 415, 288; (d) Q. Xu, M. Chandra, J. Power Source 2006, 163, 364; (e) J. M. Yan, X. B. Zhang, S. Han, H. Shioyama, Q. Xu, J. Power Source 2009, 194, 478; (f) T. Umegaki, J. M. Yan, X. B. Zhang, H. Shioyama, N. Kuriyama, Q. Xu, J. Power Source 2010, 195, 8209; (g) P. Z. Li, K. Aranishi, Q. Xu, Chem. Commun. 2012, 48, 3173.
12 (a) S. Guo, S. Dong, E. Wang, ACS Nano 2010, 4, 547; (b) C. Zhu, S. Guo, S. Dong, Adv. Mater. 2012, 24, 2326; (c) K. Aranishi, A. K. Singh, Q. Xu, ChemCatChem 2013, 5, 2248; (d) S. K. Singh, Y. Iizuka, Q. Xu, Int. J. Hydrogen Energy 2011, 36, 11794; (e) A. K. Singh, Q. Xu, ChemCatChem 2013, 5, 3000.
13 (a) A. J. Amali, K. Aranishi, T. Uchida, Q. Xu, Part. Part. Syst. Charact. 2013, 30, 888; (b) R. Yi, R. Shi, G. Gao, N. Zhang, X. Cui, Y. He, X. Liu, J. Phys. Chem. C 2009, 113, 1222; (c) D. Sun, V. Mazumder, O. Metin, S. Sun, ACS Nano 2011, 5, 6458; (d) Q. L. Zhu, J. Li, Q. Xu, J. Am. Chem. Soc. 2013, 135, 10210.
14 (a) K. Aranishi, H. L. Jiang, T. Akita, M. Haruta, Q. Xu, Nano Res. 2011, 4, 1233; (b) H. L. Wang, J. M. Yan, Z. L. Wang, Q. Jiang, Int. J. Hydrogen Energy 2012, 37, 10229.
15 (a) S. Akbayrak, P. Erdek, S. Özkar, Appl. Catal. B: Environ. 2013, 142-143, 187; (b) L. Yang, W. Luo, G. Cheng, ACS Appl. Mater. Interfaces 2013, 5, 8231.
16 J. M. Yan, X. B. Zhang, S. Han, H. Shioyama, Q. Xu, Inorg. Chem. 2009, 48, 7389.
17 M. Wen, B. Sun, B. Zhou, Q. Wu, J. Peng, J. Mater. Chem. 2012, 22, 11988.
18 M. Rakap, S. Ozkar, Int. J. Hydrogen Energy 2011, 36, 7019.
19 Z. L. Wang, J. M. Yan, Y. Ping, H. L. Wang, W. T. Zheng, Q. Jiang, Angew. Chem. Int. Ed. 2013, 52, 4406.
20 O. N. Senkov, D. B. Miracle, Mater. Res. Bull. 2001, 36, 2183.
21 (a) M. Mavrikakis, B. Hammer, J. K. Norskov, Phys. Rev. Lett. 1998, 81, 2819; (b) J. R. Kitchin, J. K. Norskov, M. A. Barteau, J. G. Chen, Phys. Rev. Lett. 2004, 93, 156801.
22 S. K. Singh, Y. Iizuka, Q. Xu, Int. J. Hydrogen Energy 2011, 36, 11794.
23 K. W. Park, J. H. Choi, B. K. Kwon, S. A. Lee, Y. E. Sung, H. Y. Ha, S. A. Hong, H. Kim, A. Wieckowski, J. Phys. Chem. B 2002, 106, 1869.

### References from Calculation Section

1 P. Hohenberg, W. Kohn, Phys. Rev. 1964, 136, B864-B871.
2 W. Kohn, L. J. Sham, Phys. Rev. 1965, 140, A1133-A1138.
3 S. J. Clark, M. D. Segall, C. J. Pickard, P. J. Hasnip, M. I. J. Probert, K. Refson, M. C. Payne, Z. Kristallogr. 2005, 220, 567-570.
4 M. D. Segall, J. D. L. Philip, M. J. Probert, C. J. Pickard, P. J. Hasnip, S. J. Clark, M. C. Payne, J. Phys.: Condens. Matter. 2002, 14, 2717-2744.
5 D. Vanderbilt, Phys. Rev. B 1990, 41, 7892-7895.
6 J. P. Perdew, K. Burke, M. Ernzerhof, Phys. Rev. Lett. 1996, 77, 3865-3868.
7 T. H. Fischer, J. Almlof, J. Phys. Chem. 1992, 96, 9768-9774.
8 H. J. Monkhorst, J. D. Pack, Phys. Rev. B 1976, 13, 5188-5192.
9 L.-Y. Gan, R.-Y. Tian, X.-B. Yang, H.-D. Lu, Y.-J. Zhao J. Phys. Chem. C 2011, 116, 745.
10 J. Greeley and M. Mavrikakis J. Phys. Chem. B 2005, 109, 3460-3471.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A trimetallic catalyst alloy comprising:
nickel, silver and palladium metal.

2. The catalyst alloy of claim 1, wherein the nickel, silver and palladium metals are present in a ratio of from about 1 to about 1 to about 1.

3. The catalyst alloy of claim 2, wherein the ratio is determined by ICP-MS.

4. The catalyst alloy of claim 1, wherein the catalyst comprises nanoparticles of from about 1 to about 10 nm.

5. The catalyst alloy of claim 1, wherein an X-ray diffraction pattern comprises a face-centered cubic (fcc) structure.

6. The catalyst alloy of claim 5, wherein no impurities are included.

7. The catalyst alloy of claim 1, further comprising the catalyst on a solid support.

8. The catalyst alloy of claim 7, wherein the solid support is an inorganic oxide carrier.

9. The catalyst alloy of claim 8, wherein the inorganic oxide carrier is a metal oxide comprising alumina, silica, titania or zirconia.

10. The catalyst alloy of claim 7, wherein the surface area of the support is at least 100 g/m² to at least 1500 g/m².

11. The catalyst alloy of claim 7, wherein the solid support is activated carbon.

12. The catalyst alloy of claim 11, wherein the activated carbon has a surface area of from about is at least 100 g/m² to at least 1500 g/m².

13. A method to produce hydrogen comprising the step of contacting ammonia borane, represented by the chemical formula NH₃BH₃, with the catalyst alloy of claim 1.

14. A method to provide a hydrogen supply comprising supplying hydrogen generated by claim 13 to a fuel cell.

15. The method of claim 14, wherein the fuel cell is a polymer electrolyte fuel cell, phosphoric acid fuel cell, or a high temperature fuel cell.

16. A method to prepare a trimetallic catalyst alloy comprising a combination of nickel, silver and palladium metal comprising the steps:
combining a nickel (II) salt, silver (II) salt and palladium (IV) salt in a solvent to provide a first mixture; and
treating the first mixture with a reducing agent to provide a second mixture which results in a trimetallic nickel, silver and palladium trimetallic catalyst.

17. The method of claim 16, further comprising treating the second mixture under ultrasonic conditions.

18. The method of claim 16, further comprising the step of adding a solid support to either the first mixture or the second mixture.

19. A method to hydrogenate olefins comprising the step:
treating an olefin with a NiAgPd catalyst as claimed in claim 1 in the presence of hydrogen.

20. The method of claim 20, wherein the catalyst alloy is supported on an inorganic oxide or a carbon support material.
